(19) United States
(12) Patent Application Publication
(10) Pub. No.: US 2021/0267797 A1
(43) Pub. Date: Sep. 2, 2021

(54) Device For Assisting In The Use Of A Device For Dispensing Liquid Product In The Form Of Drops

(71) Applicant: Nemera La Verpillière, LA VERPILLIERE (FR)

(72) Inventor: Thierry Decock, Lyon (FR)

(21) Appl. No.: 16/967,600

(22) PCT Filed: Feb. 5, 2019
(86) PCT No.: PCT/EP2019/052776

§ 371 Date: Nov. 5, 2020

(30) Foreign Application Priority Data
Feb. 6, 2018 (FR) 1850984

(51) Int. Cl. A61F 9/00; A61M 31/00

(52) U.S. Cl. CPC A61F 9/0026; A61M 31/00; A61M 2210/0662; A61M 2205/13; A61M 2205/3313; A61M 2205/3344; A61M 2205/3306; A61M 2205/3379; A61M 2205/3327; A61M 2205/50; A61M 2205/3317; A61M 2205/276

(57) ABSTRACT

The invention relates to an assistance device for assisting in the use of a device for dispensing a liquid product in the form of drops into a receiving organ of a subject, the dispensing device having a tank, the assistance device including a connector for connecting to the dispensing device, a surface bearing against the skin of the subject, arranged near an orifice for dispensing the liquid product, and means for detecting a contact between the bearing surface and the skin of the subject.

### FIELD OF THE INVENTION

The invention relates to an assistance device for assisting in the use of a device for dispensing a liquid product in the form of drops.

### BACKGROUND OF THE INVENTION

When dispensing a liquid product in the form of drops, it may be difficult to hold and use the dispensing device correctly so that the drops reach the target organ. Incorrect handling of the device could dispense drops outside the target organ, or even injure the subject.

Document WO2016/183394 describes a drop dispensing device capable of detecting colours using colour sensors and of comparing them with a range of predetermined colours to detect the presence of an eye and a possible wink. This device ensures that an eye is positioned opposite the dispensing orifice when dispensing a drop. However, it cannot guarantee that the drop actually reaches the eye since the drop may, in particular, deviate relative to the eye, even if the latter is detected by the colour sensors. In addition, it may provide an incorrect indication according to which an eye is present when the skin or an object with colours similar to those of an eye is positioned in front of the dispensing orifice.

The invention aims in particular to provide an assistance device for assisting in the use of a dispensing device which simplifies its handling and which ensures that a drop reaches the target organ.

### SUMMARY OF THE INVENTION

The invention therefore relates in particular to an assistance device for assisting in the use of a device for dispensing a liquid product in the form of drops into a receiving organ of a subject, the dispensing device comprising a tank, the assistance device comprising:
means for connecting to the dispensing device,
a surface bearing against the skin of the subject, which is arranged near an orifice for dispensing the liquid product,
means for detecting a contact between the bearing surface and the skin of the subject.

Due to the presence of the bearing surface, the position of the dispensing orifice with respect to the receiving organ, and in particular the distance between the dispensing orifice and the receiving organ, can be fixed. This limits the risks of the drop being diverted, by guaranteeing that it is always dispensed at an optimum distance between the dispensing orifice and the receiving organ, and thus that it actually reaches the receiving organ. To position the dispensing device with respect to the receiving organ, the subject presses the bearing surface against the skin around the receiving organ and no longer needs to aim approximately for the receiving organ, which is the case when there is no bearing surface. Thus, the bearing surface helps the subject to position and handle the dispensing device since the subject simply needs to press the bearing surface against the skin of the receiving organ, or around the receiving organ, to dispense a drop into the receiving organ at a suitable distance.

In addition, the means for detecting a contact between the bearing surface and the skin of the subject can be used to indicate to the assistance device that the dispensing device is a priori correctly positioned and therefore ready to dispense the liquid. This guarantees in particular that the drop is actually dispensed into the receiving organ to avoid, for example, waste or an error in the number of drops to be dispensed. When it detects the contact, the assistance device can process the information in numerous ways, one example being to allow the dispensing or to inform the subject that the dispensing can be triggered.

Note that the receiving organ can be an eye, ear, part of the skin or any other organ of a living being. The subject may be a human or an animal.

The assistance device may further comprise one or more of the following characteristics, taken alone or in combination.

The assistance device comprises separation means configured to preserve a predetermined distance between the bearing surface and the drop dispensing orifice. The distance between the bearing surface and the dispensing orifice represents the distance that the drop must travel to reach the receiving organ. This distance is preferably relatively small, of the order of a few centimeters, for example 1 to 3 cm, to avoid any risk of the drop being diverted. In some cases, it avoids direct contact between the dispensing orifice and the target organ to avoid injuring the subject and possibly avoid contaminating the dispensing orifice. By configuring a predetermined distance, the dispensing device therefore becomes easy and safe to use, guaranteeing that the drop is dispensed into the receiving organ.

The assistance device comprises means for detecting a colour of an area arranged opposite the bearing surface and/or the drop dispensing orifice, and possibly an information processing system to compare the colour detected with at least one predetermined colour. Thus, a characterizing colour of the receiving organ can be detected, or the colour detected can be compared with one or more predetermined characterizing colours of the receiving organ. This ensures that the dispensing orifice is positioned opposite the receiving organ, which is particular advantageous if the receiving organ has a colour different from the part of the subject surrounding the receiving organ. In particular, when the receiving organ is an eye, this can be used to check that the eye is open, since the potential colours of an open are different, for example, from the colour of an eyelid. The colour detection means can also be used to validate the information obtained by the means for detecting a contact and help to ensure, for example, that the drop is dispensed into an open eye. According to one example, the detection means can be configured to detect one or more colours of the area arranged opposite the bearing surface and/or the drop dispensing orifice, and then it with a range of predetermined colours using the processing system. They can further or alternatively provide a positive or negative signal depending on the colour detected.

The assistance device comprises means for locking the dispensing of product adapted to only allow activation of the dispensing device depending on the contact detected and/or a colour detected. Advantageously, the locking means can block the dispensing of a drop when there is no contact and/or no colour detected, and wait until the contact and/or the colour is detected to automatically trigger the dispensing. For example, when the receiving organ is an eye, the locking means can block the dispensing of a drop when the eye is closed, even if the subject activates the dispensing, and wait for the eye to open to trigger the dispensing. The locking means can lock the dispensing of product depending on the information provided by the means for detecting a contact or by colour detection means, or both. It is understood that the case where activation is only allowed once a contact and a colour are detected is particularly advantageous since it ensures in a particularly reliable manner that the dispensed drop is actually received in the receiving organ. “Activation of the dispensing device” means the fact that a subject exerts a pressure, directly or by means of the assistance device, on the dispensing device to dispense a drop of liquid.

The assistance device comprises optical means intended to be arranged near the drop dispensing orifice, and configured to provide information on the dispensing of a drop of the liquid product by the dispensing device. More precisely, the optical means can be used to detect the presence or absence of a drop near the dispensing orifice, providing information on the formation of a drop and/or the absence of a drop, therefore information on the dispensing of the drop. Thus, by combining them with the means for detecting a contact, we obtain an additional guarantee that the drop will actually be dispensed into the targeted organ. The optical means preferably detect the presence of a drop by detecting a disturbance of an optical signal caused by this presence.

The assistance device comprises means for measuring the inclination, configured to provide information on the inclination of the dispensing device connected to the assistance device, and a system for processing information on the dispensing of a drop and on the inclination of the dispensing device connected to the assistance device to provide information on the amount of liquid product dispensed. We see that the inclination of the dispensing device while dispensing a drop affects the volume of the dispensed drop. Generally in fact, the more the axis of the dispensing orifice is directed perpendicular to the surface of the receiving organ, the larger the drop. Thus, by measuring the inclination and by providing this information to the processing system, the latter can estimate the volume of the dispensed drop and possibly calculate the volume remaining in the dispensing device.

The assistance device comprises means for measuring the weight of the dispensing device connected to the assistance device, configured to provide information on the amount of liquid product remaining in the dispensing device. By weighing the dispensing device, we obtain the weight of liquid product remaining and, knowing its relative density, we can obtain the volume of liquid product remaining which can possibly be converted in a number of remaining drops. Knowing the volume of remaining product by weighing before and after the dispensing of a drop, the amount of product dispensed can also be determined. The value obtained can also be compared with that obtained by other means, for example by measuring the inclination of the assistance device, and thus perform a double-check of the estimated amount of dispensed product.

The assistance device comprises means for adjusting the bearing surface, so as to be able to vary the distance maintained between the dispensing orifice and at least a part of the bearing surface. The user can therefore adapt the dispensing distance according to their requirements and comfort, while guaranteeing correct dispensing of a drop into the receiving organ since, after adjustment, the distance between the bearing surface and the dispensing orifice is kept fixed. The adjustment means may comprise, for example, a worm screw or a spring combined with locking means or a replaceable end piece.

The means for detecting a contact comprise light detection means, configured to detect a variation in the light intensity over a part of the bearing surface. When the subject presses the bearing surface against the skin, since the skin is opaque, it prevents light from falling on at least a part of the bearing surface, causing a variation in the intensity of the light on this part of the bearing surface. Thus, by detecting this variation, the contact between the skin and the bearing surface is detected. To detect the variation in the light intensity, the light detection means may comprise light sensors arranged on the surface of the bearing surface, preferably under a light-permeable part of the bearing surface. The light detection means may also be arranged in a tunnel opening out onto the bearing surface or onto a light-permeable area of the bearing surface. “Light” means a light having at least one wavelength within the visible spectrum. The light detection means may comprise several light sensors distributed angularly around the dispensing orifice to evaluate the intensity of the light at several positions around the dispensing device, for example pairs of light sensors diametrically opposed relative to the dispensing orifice.

The means for detecting a contact comprise at least one electrode connected to an electrical circuit, the electrode being arranged on the bearing surface, the electrical circuit being adapted to detect a modification in at least one value chosen from the voltage, current and electrical resistance, when the skin of a subject is in contact with the electrode. Since the skin is an electrical conductor of given electrical resistance, when it is placed in contact with the bearing surface, the electrical circuit detects a modification characteristic of the skin's electrical resistance indicating that the skin is in contact with the bearing surface. Advantageously, the electrodes are capacitive sensors. Preferably, two or more pairs of electrodes are provided for greater accuracy.

The means for detecting a contact comprise means for detecting a minimum pressure exerted on the bearing surface. When a subject places the receiving organ within reach of the dispensing orifice, the subject presses the skin against the bearing surface and exerts a pressure (i.e. force) on this bearing surface. Detection of this pressure provides information not only on the presence of the contact but also on a press exerted. The fact of pressing allows the drop to be dispensed in a more stable or assured manner. The means for detecting a minimum pressure comprise, for example, a pressure sensor configured to provide preferably quantitative information on the pressure exerted on the bearing surface.

The means for detecting a minimum pressure comprise a first air chamber that deforms when the bearing surface is pressed against the skin of the subject, and at least one pressure sensor adapted to be activated when the pressure of the first air chamber reaches a predetermined threshold. The pressure detected can be the air pressure in the first air chamber or a contact pressure (i.e. force) applied to the first air chamber. In the first case, the skin pressing against the bearing surface may cause a variation in the air pressure inside the first air chamber until the predetermined threshold is reached. In the second case, the skin pressing against the bearing surface may cause a contact between the bearing surface and the means of detecting a minimum pressure by deformation of the first air chamber until the pressure or force reaches the predetermined threshold. The first air chamber may comprise an appendage to simplify the contact between the bearing surface and the means for detecting a minimum pressure. It is understood that the air chamber is airtight and that the air chamber casing is advantageously elastically deformable.

The means for detecting a minimum pressure comprise a second air chamber of volume less than that of the first air chamber, the second air chamber being arranged between the first air chamber and the pressure sensor so that the forces are concentrated on the pressure sensor. The second air chamber is used to distribute a local press of the skin against the bearing surface more homogeneously. The means for detecting a minimum pressure are therefore more sensitive and less dependent on the position where the press is exerted. The pressure in the first air chamber is then measured via the second air chamber.

The means for detecting a minimum pressure comprise two electrodes connected to a source of electricity and an electrically conductive part mounted so as to be able to move between an open circuit position, in which an electric circuit formed by the source of electricity and the electrodes is open, and a closed circuit position, in which the electrically conductive part electrically connects the two electrodes, the electrically conductive part moving between the two positions when a minimum pressure is exerted on the bearing surface. Thus, when the bearing surface is pressed against the skin, the pressure applied moves the electrically conductive part towards the closed circuit position. In this case, the contact between the bearing surface and the skin is detected by the flow of an electric current. The electrically conductive part can move between the two positions, for example by elastic deformation or by translation.

The means for detecting a minimum pressure comprise a return element arranged between the electrically conductive part and the electrodes and tending to return the electrically conductive part to the open circuit position, the return element being for example a spring or an air chamber arranged between the electrically conductive part and the electrodes. The skin pressing against the bearing surface may deform the return element, thereby moving the electrically conductive part towards its closed circuit position, indicating detection of the contact. When the skin is no longer in contact with the bearing surface, the return element takes up its rest position, moving the electrically conductive part back towards its open circuit position.

The invention further relates to a kit for dispensing a liquid product into a receiving organ of a subject, comprising a device for dispensing liquid product and an assistance device as described previously. The dispensing kit dispenses a drop of liquid into the receiving organ simply and precisely. Preferably, the assistance device and the dispensing device are separate devices, attached to each other and removable, but they could form a single unit and be made of the same material.

### BRIEF DESCRIPTION OF THE DRAWINGS

We will now describe particular embodiments of the invention given as non-limiting examples referring to the attached figures, on which:

FIG. 1 is a set of two views, with on the left a perspective exploded view and on the right a perspective assembled view of a dispensing device connected to an assistance device according to a first embodiment,

FIG. 2 is a set of views similar to that of FIG. 1 of a dispensing device connected to an assistance device, according to a second embodiment,

FIG. 3A is a set of views similar to that of FIG. 1 of a dispensing device connected to an assistance device, according to a third embodiment,

FIG. 3B is a schematic longitudinal cross-sectional view (the longitudinal direction corresponding to that of the drop dispensing axis) of an upper part of the assistance device according to a variant of the third embodiment,

FIG. 3C is a schematic longitudinal cross-sectional view of an upper part of the assistance device according to another variant of the third embodiment,

FIG. 4A is a set of views similar to that of FIG. 1 of a dispensing device connected to an assistance device, according to a fourth embodiment,

FIG. 4B is a schematic longitudinal cross-sectional view of an upper part of the assistance device according to the fourth embodiment,

FIG. 5A is a set of views similar to that of FIG. 1 of a dispensing device connected to an assistance device, according to a fifth embodiment,

FIG. 5B is a schematic longitudinal cross-sectional view of an upper part of the assistance device according to the fifth embodiment.

### DETAILED DESCRIPTION OF THE INVENTION

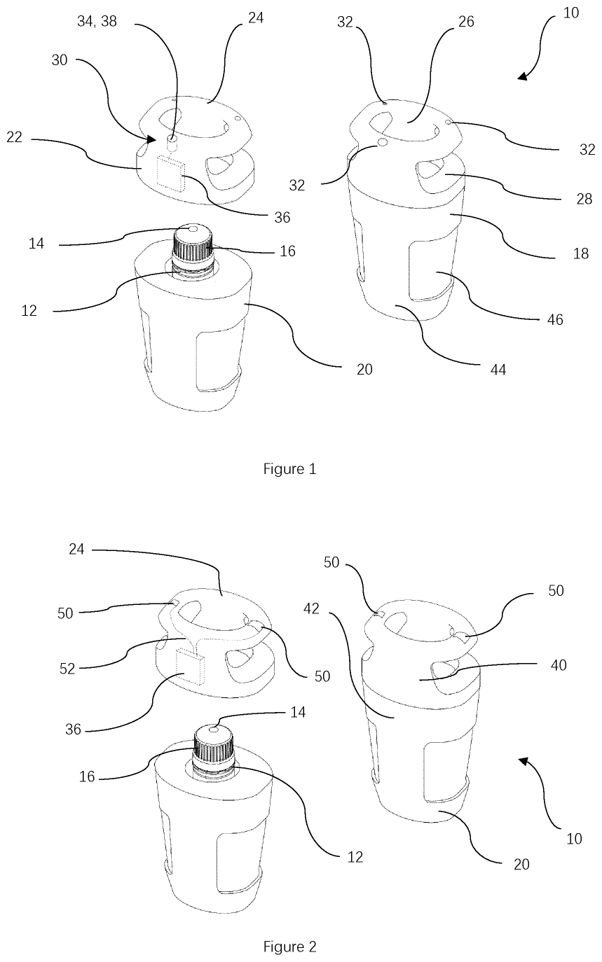

FIG. 1 illustrates an assistance device 10 for assisting in the use of a device 12 for dispensing a liquid product in the form of drops into a receiving organ of a subject according to a first embodiment.

The dispensing device 12 comprises in this case a tank and a dispensing end piece provided with an orifice 14 for dispensing drops protected by a cap 16, for example screwed onto the dispensing end piece. On the figures, the reference 14 indicates the position of the dispensing orifice 14 covered by the cap 16. The assistance device 10 comprises means 18 arranged therein for connecting to the dispensing device 12 so that the assistance device 10 and the dispensing device 12 form an integral assembly. The connection means 18 may comprise, for example, means for clipping the tank into the assistance device 10. In a particular variant, the dispensing end piece provided with the dispensing orifice 14 could form part of the assistance device 10, attached to the tank of the dispensing device 12 when connecting the assistance device 10 and the dispensing device 12.

The assistance device 10 comprises a main body 20 in which the dispensing device 12 is placed, and a bearing structure 22 comprising a bearing surface 24 to be pressed against the user's skin when dispensing drops into a receiving organ, for example an eye. The bearing structure 22 is removably mounted on the main body 20 in order to position and connect the dispensing device 12 inside the assistance device 10, possibly by means of a hinge, or being fully detachable. The bearing structure 22 can be designed to be flexible enough, in particular at the bearing surface 24, to rest comfortably against the users skin and adapt to the different reliefs near the target organ, and/or rigid enough to provide support when pressing and impose a predetermined distance between the dispensing orifice 14 and the target organ. The bearing surface 24 comprises an axial orifice 26 intended to allow drops of liquid product to fall from the dispensing orifice 14 towards the receiving organ. The bearing structure 22 optionally comprises through-openings 28 on two opposite sides and at its end, in particular to prevent the user's eye from being in the dark when the assistance device 10 is applied against the user's skin around the eye, or to make some parts of the bearing surface 24 more flexible so as to be more comfortable for the subject.

The assistance device 10 comprises separation means configured to preserve a predetermined distance between the bearing surface 24 and the dispensing orifice 14. The separation means may be a rigid part of the bearing structure 22 which keeps the bearing surface 24 away from the dispensing orifice 14. Since the bearing structure 22 is removably mounted on the main body 20, it can act as means for adjusting the bearing surface 24, so as to be able to vary the distance maintained between the dispensing orifice 14 and at least a part of the bearing surface 24, by changing at least a part of the bearing structure 22. In a variant, the adjustment means may comprise a worm screw or a flexible element which can lock at predetermined distances.

The assistance device 10 comprises means for detecting a contact between the bearing surface and the skin of the subject. In this case, the means for detecting a contact comprise light detection means 30, configured to detect a variation in the light intensity over a part of the bearing surface 24. In this example, the light detection means 30 comprise three tunnels 32 opening out onto the bearing surface 24, distributed angularly on the bearing surface 24. Each channel 32 contains a light sensor 34 preferably connected to a system 36 for processing the information provided by the light sensors 34. Thus, when a subject places their skin against the bearing surface 24, the light no longer passes through the tunnels 32. The light sensors 34 therefore detect a variation in the light intensity, indicating that the liquid is ready to be dispensed. Note that the number of tunnels 32 is not limited to three. They can be configured and positioned as required.

In a variant, the light detection means 30 comprise light sensors 34 arranged directly on the bearing surface 24 or under a light permeable part thereof. Thus, at least a part of the bearing surface 24 can be made of a light permeable material that can be detected by the light sensors 34.

The assistance device 10 further comprises means 38 (shown on FIG. 1) for detecting a colour of an area arranged opposite the bearing surface and/or the drop dispensing orifice 14. Like the light detection means 30, the means 38 for detecting a colour comprise one or more colour sensors arranged on or near the bearing surface 24 and configured to be directed towards the receiving organ when the assistance device 10 is pressed against the skin of the subject, adapted to detect one or more colours determined as being characteristic of the receiving organ and to provide a positive or negative signal depending on the detection. In a variant, the colour sensors detect one or more colours of the object placed opposite them and send the information to the processing system 36 which compares them with at least one colour or a range of predetermined colours characteristic of the receiving organ.

The assistance device 10 comprises means for locking the dispensing of product adapted to only allow activation of the dispensing device 12 depending on the contact detected and/or a colour detected. The locking means comprise, for example, a valve arranged in the dispensing end piece, adapted to block the dispensing of liquid if a contact between the skin and the bearing surface 24 is not detected by the light detection means 30 and/or if a predetermined colour is not detected by the means 38 for detecting a colour.

The assistance device 10 comprises optical means 40 intended to be arranged near the dispensing orifice 14, and configured to provide information on the dispensing of a drop of the liquid product by the dispensing device 12. The optical means 40 comprise a transmitter and a receiver of an optical signal, configured to detect the presence of a drop disturbing the optical signal and to measure the duration of this presence. The transmitter comprises, for example, infrared-emitting diodes and the receiver comprises for example phototransistors capable of detecting infrared rays. The transmitter and the receiver detect the presence of a drop passing through the optical signal when the optical rays are disturbed, for example by a variation in the intensity of the rays. The transmitter and the receiver are preferably located at a distance of between 1 and 3 mm, preferably 2 mm, from the dispensing orifice 14.

The assistance device 10 further comprises means 42 for measuring the inclination, configured to provide information on the inclination of the dispensing device 12 connected to the assistance device 10. The means 42 for measuring the inclination comprise, for example, an inclinometer such as an electronic gyroscope or an accelerometer. The inclinometer is preferably placed in the main body 20, for example in an area intended to be placed near the dispensing end piece.

The assistance device 10 comprises means 44 for measuring the weight of the dispensing device 12 connected to the assistance device 10, configured to provide information on the amount of liquid product remaining in the dispensing device 12. The means 44 for measuring the weight comprise a weight sensor, for example of the Force Sensing Resistor (FSR) type, arranged under the tank to weigh the dispensing device 14 and deduce the weight, and therefore the volume, of the amount of liquid remaining in the tank. The weight sensor 44 can be located under or above the tank. In another variant, the assistance device 10 comprises several weight sensors which are arranged around the tank to measure the weight of the dispensing device 12 connected to the assistance device 10, regardless of its inclination.

The optical means 40, the means 42 for measuring the inclination and the means 44 for measuring the weight transmit the information to the processing system 36 which uses said information and/or combines it with other information, to estimate the amount of liquid product dispensed, more particularly the volume of the dispensed drop. To do this, the processing system 36 first calculates a theoretical drop volume, using information on the viscosity of the liquid product and/or the geometric characteristics of the dispensing orifice 14, or even on other characteristics of the dispensing end piece and/or of the dispensing device 12. It then weight this theoretical volume by taking into account information provided by the above means, for example by calculating a coefficient related to one or more parameters such as the magnitude of the activation pressure applied by the user on the tank to cause the formation of the drop, the variation profile of this activation pressure over time, the time during which the optical signal provided by the optical means 40 is disturbed, the inclination of the dispensing device 12 connected to the assistance device 10 with respect to a horizontal axis and the measurement of the weight of the dispensing device 12.

The assistance device 10 also comprises a gripping area 46 intended in this case to allow the user both to grip and to press in order to dispense the liquid product. The gripping area 46 is arranged on two opposite sides of the main body 20. An activation pressure exerted on the gripping area 46 is transmitted to the tank of the dispensing device 12 to dispense the liquid product. The gripping area 46 may be made from a different material, in particular more flexible than that of the rest of the main body 20. It may also comprise reliefs to facilitate gripping by the user. Furthermore, due to the presence of the gripping area 46, the assistance device 10 increases the user's gripping area and the activation pressure area on the tank compared with that of the dispensing device 12 alone, which is particularly advantageous for users with neuromuscular diseases.

FIG. 2 illustrates an assistance device 10 for assisting in the use of a device 12 for dispensing a liquid product in the form of drops into a receiving organ of a subject according to a second embodiment. The assistance device 10 of FIG. 2 differs from that of FIG. 1, mainly by the means for detecting a contact. The rest of the structure is substantially similar and is not described. The elements in common are identified by the same numerical references in the remainder of the description.

In this second embodiment, the means for detecting a contact comprise two electrodes 50 connected to an electrical circuit 52. The electrodes 50 are arranged diametrically opposite each other on the bearing surface 24. They can be placed under an electrically conductive covering layer to improve the aesthetic appearance. Advantageously, the electrodes 50 are capacitive sensors configured to generate an electric current in the circuit 52 when an object having electrical properties, in particular an electrical resistance, similar to those of the skin.

Thus, the electrical circuit 52 is adapted to detect a modification in at least one value chosen from the voltage, current and electrical resistance when the skin of a subject is in contact with the electrodes 50. The electrical circuit 52 therefore comprises conducting wires, a source of electricity and other electronic components such as a voltmeter or an ammeter. The electrical circuit 52 is connected to the processing system 36 which receives information on the contact between the skin and the bearing surface 24 to trigger or not dispensing of liquid.

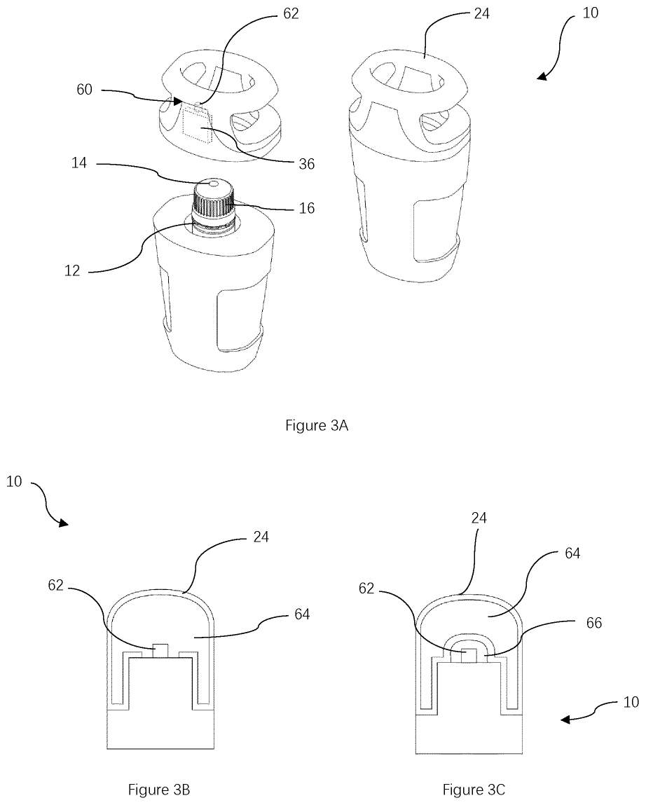

FIGS. 3A, 3B and 3C illustrate an assistance device 10 for assisting in the use of a device 12 for dispensing a liquid product in the form of drops into a receiving organ of a subject according respectively to a third embodiment and two variants of this third embodiment. The assistance device 10 of FIGS. 3A, 3B and 3C differs from that of FIG. 1, mainly by the means for detecting a contact.

The means for detecting a contact of FIG. 3A comprise means 60 for detecting a minimum pressure exerted on the bearing surface 24. The means 60 for detecting a minimum pressure comprise in this case a pressure sensor 62 having a branch extending up to the bearing surface 24 or up to contact with a layer of deformable material of the bearing surface 24. When a subject presses the skin against a bearing surface 24, the subject exerts a pressure (i.e. force), greater than the predefined minimum pressure on this bearing surface. Detection of this pressure provides information on the presence of the contact between the skin and the bearing surface 24.

The means for detecting a contact, shown on FIG. 3B, also comprise a first deformable air chamber 64 between the bearing surface 24 and the pressure sensor 62. The first air chamber 64 comprises a tight elastic casing filled with air or another type of fluid. The casing can support the bearing surface 24 on an outer face. The first air chamber 64 deforms when the skin of the subject is pressed against the bearing surface 24. When the air pressure of the first air chamber 64 exceeds a predetermined threshold, the pressure sensor 62 is activated and provides information on the correct positioning of the receiving organ with respect to the dispensing orifice 14. In this case, the pressure sensor 62 is configured to detect the air pressure. Alternatively, the pressure sensor 62 is activated when the bearing surface 24, under the pressure against the skin, moves towards the pressure sensor 62 by deformation of the first air chamber 64 until a pressure greater than the predetermined threshold is exerted. In this case, the pressure sensor 62 is configured to detect a contact pressure. (i.e. force). The contact between the bearing surface 24 and the pressure sensor 62 can be assisted by an appendage arranged in the first air chamber 64.

The means for detecting a contact, shown on FIG. 3C, comprise a second air chamber 66 of volume less than that of the first air chamber 64. The second air chamber 66 is arranged between the first air chamber 64 and the pressure sensor 62 so that the forces are concentrated on the latter. When the subject positions the receiving organ opposite the dispensing orifice 14, the skin presses against the bearing surface 24, first deforming the first air chamber 64 then the second air chamber 66 to exert a pressure on the pressure sensor 62 directly by contact or by compressing the air inside the second air chamber 66 containing the pressure sensor 62. The assistance device is adapted to process information when the pressure exceeds the predetermined threshold, for example to allow the dispensing of liquid product.

In the three figures, the means 60 for detecting a minimum pressure are connected to the processing system 36 to provide it with information on the correct positioning of the receiving organ opposite the dispensing orifice 14 and/or quantitative information on the pressure exerted on the bearing surface 24.

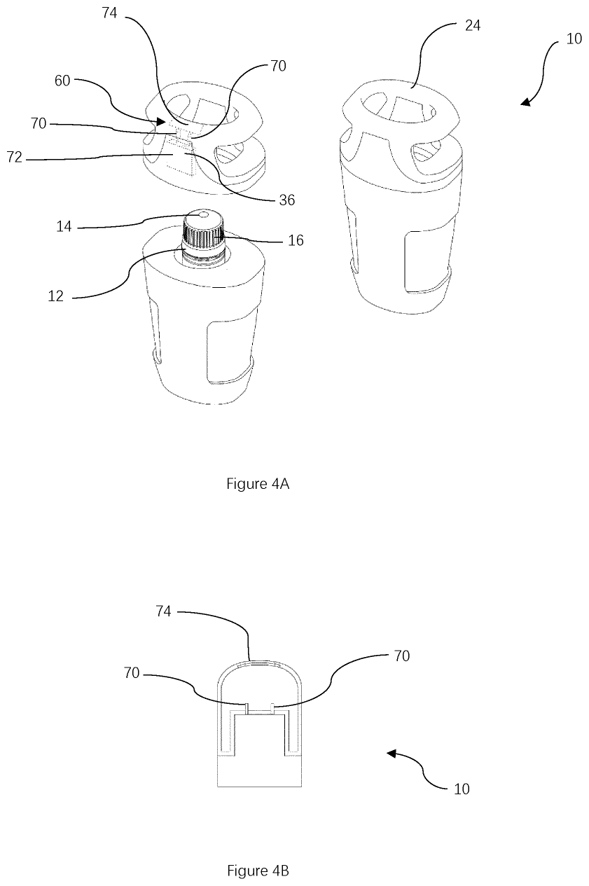

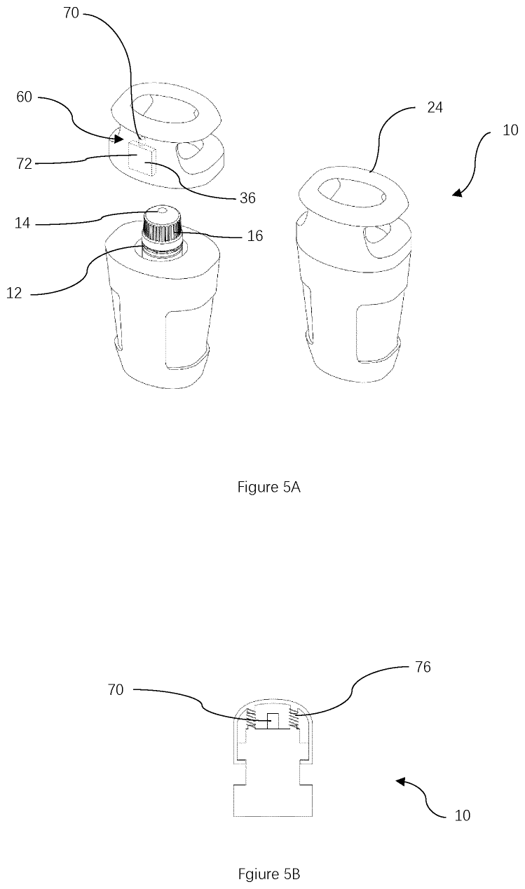

FIGS. 4A and 4B illustrate an assistance device 10 for assisting in the use of a device 12 for dispensing a liquid product in the form of drops into a receiving organ of a subject according to a fourth embodiment. The assistance device 10 of FIGS. 4A and 4B differs from that of FIG. 1, mainly by the means for detecting a contact.

The means 60 for detecting a minimum pressure comprise two electrodes 70 connected to a source of electricity 72 and an electrically conductive part 74. The electrodes 70 are placed in the bearing structure 22, away from the bearing surface 24. The electrically conductive part 74, for example a metal plate, is connected to at least a part of the bearing surface 24. The electrically conductive part 74 is mounted so as to be able to move between an open circuit position, in which an electric circuit formed by the source of electricity and the electrodes is open, and a closed circuit position, in which the electrically conductive part electrically connects the two electrodes 70. When the subject places the receiving organ opposite the dispensing orifice 14, the subject presses the skin against the bearing surface 24 which, due to the pressure and being flexible, deforms and moves the electrically conductive part 74, to which it is connected, from the open circuit position to the closed circuit position. When the circuit is closed, current flows therein and detection of this current indicates the contact and the correct positioning of the receiving organ which is ready to receive the dispensing of liquid.

FIGS. 5A and 5B illustrate an assistance device 10 for assisting in the use of a device 12 for dispensing a liquid product in the form of drops into a receiving organ of a subject according to a fifth embodiment. The assistance device 10 of FIGS. 5A and 5B is a variant of that shown on FIGS. 4A and 4B.

The means 60 for detecting a minimum pressure comprise a return element 76, for example a spring, between each of the electrodes 70 and the electrically conductive part 74. The return element 76 deforms, for example by compressing, when the electrically conductive part 74 moves closer to the electrodes 70 under the pressure of the skin pressing on the bearing surface 24. Once the skin has left the bearing surface 24, the pressure disappears, thereby releasing the return element 76 which returns to its undeformed position, taking with it the electrically conductive part 74.

The invention is not limited to the embodiments described and other embodiments will be clearly apparent to those skilled in the art.

1. An assistance device for assisting in the use of a device for dispensing a liquid product in the form of drops into a receiving organ of a subject, the dispensing device comprising a tank, the assistance device comprising:
a connector for connecting to the dispensing device,
a surface bearing against the skin of the subject, arranged near an orifice for dispensing the liquid product, and
a contact detector for detecting a contact between the bearing surface and the skin of the subject.

2. The assistance device according to claim 1, comprising a separator configured to preserve a predetermined distance between the bearing surface and the drop dispensing orifice.

3. The assistance device according to claim 1, further comprising a colour detector for detecting a colour of an area arranged opposite the bearing surface and/or the drop dispensing orifice, and an information processing system to compare the colour detected with at least one predetermined colour.

4. The assistance device according to claim 1, further comprising a locking member for locking the dispensing of product adapted to only allow activation of the dispensing device depending on the contact detected and/or a colour detected.

5. The assistance device according to claim 1, further comprising an optical device intended to be arranged near the drop dispensing orifice, and configured to provide information on the dispensing of a drop of the liquid product by the dispensing device.

6. The assistance device according to claim 5, further comprising a metering device for measuring the inclination, configured to provide information on the inclination of the dispensing device connected to the assistance device, and a system for processing information on the dispensing of a drop and on the inclination of the dispensing device connected to the assistance device to provide information on the amount of liquid product dispensed.

7. The assistance device according to claim 1, comprising a metering device for measuring the weight of the dispensing device connected to the assistance device configured to provide information on the amount of liquid product remaining in the dispensing device.

8. The assistance device according to claim 1, comprising an adjusting member for adjusting the bearing surface, so as to be able to vary the distance maintained between the dispensing orifice and at least a part of the bearing surface.

9. The assistance device according to claim 1, wherein the contact detector comprises a light detector configured to detect a variation in the light intensity over a part of the bearing surface.

10. The assistance device according to claim 1, wherein the contact detector comprises at least one electrode connected to an electrical circuit, the electrode being arranged on the bearing surface, the electrical circuit being adapted to detect a modification in at least one value chosen from the voltage, current and electrical resistance, when the skin of a subject is in contact with the electrode.

11. The assistance device according to claim 1, wherein the contact detector detects a minimum pressure exerted on the bearing surface.

12. The assistance device according to claim 11, wherein the contact detector comprises a first air chamber that deforms when the bearing surface is pressed against the skin of the subject, and at least one pressure sensor adapted to be activated when the pressure of the first air chamber reaches a predetermined threshold.

13. The assistance device according to claim 12, wherein the contact detector comprises a second air chamber of volume less than that of the first air chamber (64), the second air chamber being arranged between the first air chamber and the pressure sensor so that the forces are concentrated on the pressure sensor.

14. The assistance device according to claim 11, wherein the contact detector comprises two electrodes connected to a source of electricity and an electrically conductive part mounted so as to be able to move between an open circuit position, in which an electric circuit formed by the source of electricity and the electrodes is open, and a closed circuit position, in which the electrically conductive part electrically connects the two electrodes, the electrically conductive part moving between the two positions when a minimum pressure is exerted on the bearing surface.

15. The assistance device according to claim 14, wherein the contact detector further comprises a return element arranged between the electrically conductive part and the two electrodes and tending to return the electrically conductive part to the open circuit position, the return element being a spring or an air chamber arranged between the electrically conductive part and the electrodes.

16. A kit for dispensing a liquid product into a receiving organ of a subject, comprising a device for dispensing liquid product and an assistance device according to claim 1.
